(19) United States
(12) Patent Application Publication
(10) Pub. No.: US 2023/0170085 A1
(43) Pub. Date: Jun. 1, 2023

(54) DATA PROVENANCE, LOCALIZATION, AND ANALYSIS FOR PERSONAL DATA COLLECTED IN A PRIVATE ENTERPRISE NETWORK

(71) Applicant: Nokia Solutions and Networks Oy, Espoo (FI)

(72) Inventors: Mohammad Riaz KHAWER, Wayland, MA (US); Kiti Meerit Irmeli MULLER, Helsinki (FI); Harri LINDHOLM, Helsinki (FI)

(21) Appl. No.: 17/997,584

(22) PCT Filed: May 18, 2021
(86) PCT No.: PCT/FI2021/050359

§ 371 Date: Oct. 31, 2022

Related U.S. Application Data
(60) Provisional application No. 63/029,102, filed on May 22, 2020.

(51) Int. Cl. G16H 40/67; G16H 50/20

(52) U.S. Cl. CPC G16H 40/67; G16H 50/20; G16H 20/00

(57) ABSTRACT

An edge cloud network includes one or more base stations that support wireless communication with a plurality of sensors. The edge cloud network also includes a core network that stores data collected using the plurality of sensors. The edge cloud network further includes a machine learning (ML) analytics server configured to analyze the data collected using the plurality of sensors. The edge cloud network implements sensor data provenance to ensure integrity and localization of the data within the edge cloud network. The base station supports wireless communication in at least one of a licensed spectrum, a shared Citizens Broadband Radio Service (CBRS) spectrum, an unlicensed spectrum, and an opportunistically available licensed spectrum.

### BACKGROUND

The development of high-speed wireless connections (e.g., according to Fifth Generation, 5G, standards), the increasing analytical power of machine learning (ML) techniques, and the availability of a vast array of sensors are converging to produce rapid changes in vertical markets such as healthcare and human performance. The first wave of connected sensors for monitoring individuals (and their environments) use contention-based services in unlicensed frequency bands, such as Bluetooth or Wi-Fi, to transmit data to the network. This approach has several drawbacks. For example, many sensors are tethered to smart phones owned or operated by the individual, which constrains use of the sensors to contexts in which the individual has a smart phone available, as well as reducing the reliability and security of the system. For another example, the conventional contention-based services support lower bandwidths, lower reliability, and lower quality-of-service (QoS) relative to cellular technologies such as Fourth Generation (4G) Long Term Evolution (LTE) or 5G. Connecting the sensors directly to cellular networks requires that the data be transmitted to a core network that is beyond the control of the enterprise that is collecting the data, which increases the risk that privacy or security of the data will be compromised.

### SUMMARY OF EMBODIMENTS

The following presents a simplified summary of the disclosed subject matter in order to provide a basic understanding of some aspects of the disclosed subject matter. This summary is not an exhaustive overview of the disclosed subject matter. It is not intended to identify key or critical elements of the disclosed subject matter or to delineate the scope of the disclosed subject matter. Its sole purpose is to present some concepts in a simplified form as a prelude to the more detailed description that is discussed later.

In some embodiments, an edge cloud network is provided. The edge cloud network includes at least one base station that supports wireless communication with a plurality of sensors, a core network that stores data collected using the plurality of sensors; and a machine learning (ML) analytics server configured to analyze the data collected using the plurality of sensors, wherein the edge cloud network implements sensor data provenance to ensure integrity and localization of the data within the edge cloud network.

In some embodiments, the at least one base station supports wireless communication in at least one of a licensed spectrum, a shared Citizens Broadband Radio Service (CBRS) spectrum, an unlicensed spectrum, and an opportunistically available licensed spectrum.

In some embodiments, the plurality of sensors includes a first subset configured to monitor characteristics of at least one human and a second subset configured to monitor environmental factors proximate the at least one human.

In some embodiments, the first subset of the plurality of sensors is configured to monitor biomarkers comprising at least one of counts of eye blinks, an electrocardiogram, pulse plethysmography, body temperature, and blood pressure, and the second subset of the plurality of sensors is configured to monitor at least one of a light level, an ambient noise level, and an ambient temperature.

In some embodiments, the ML analytics server is configured to identify correlations between the biomarkers and track the correlated biomarkers.

In some embodiments, the ML analytics server is configured to correlate the monitored characteristics of the at least one human with the monitored environmental factors proximate the at least one human.

In some embodiments, the ML analytics server is configured to generate, based on the monitored characteristics of the at least one human or the monitored environmental factors, feedback indicating a schedule for at least one of administering medications, performing rehabilitation activities, meals, and sleep, and wherein the generated feedback is provided to the at least one human.

In some embodiments, the plurality of sensors comprises a video camera configured to monitor motion or behavior of the at least one human.

In some embodiments, the plurality of sensors is configured to perform concurrent imaging of body organs in at least one human using at least one of structure measurements, electro-neurophysiological measurements, and measurements related to metabolism, neurohumoral physiology, and circulation-related physiology.

In some embodiments, the ML analysis server is configured to perform real-time analysis on the data collected during the concurrent imaging of the body organs by the plurality of sensors, and the ML analysis server is configured to generate feedback for at least one remote user based on the real-time analysis.

In some embodiments, the remote user reconfigures the plurality of sensors based on the feedback.

In some embodiments, the edge cloud network is configured to provide at least one of blinded, masked, or anonymized biomarker data and environmental data to a regional cloud.

In some embodiments, a method for implementation in an edge cloud network is provided. The method includes establishing sensor data provenance to ensure integrity and localization of data collected by a plurality of sensors within the edge cloud network, collecting, using the plurality of sensors, data associated with at least one human conveying the data to at least one base station within the edge cloud network, storing, at a core network implemented in the edge cloud network, the data collected using the plurality of sensors, and analyzing, using a machine learning (ML) analytics server implemented in the edge cloud network, the data collected using the plurality of sensors.

In some embodiments, conveying the data to the at least one base station includes conveying the data via at least one of a licensed spectrum, a shared Citizens Broadband Radio Service (CBRS) spectrum, an unlicensed spectrum, and an opportunistically available licensed spectrum.

Some embodiments of the method include monitoring, using a first subset of the plurality of sensors, characteristics the at least one human and monitoring, using a second subset of the plurality of sensors, environmental factors proximate the at least one human.

In some embodiments, monitoring the characteristics of the at least one human includes monitoring biomarkers comprising at least one of counts of eye blinks, an electrocardiogram, pulse plethysmography, body temperature, and blood pressure, and wherein monitoring the environmental factors comprises monitoring at least one of a light level, an ambient noise level, and an ambient temperature.

Some embodiments of the method include identifying, using the ML analytics server, correlations between the biomarkers and tracking, using the ML analytics server, the correlated biomarkers.

Some embodiments of the method include correlating, using the ML analytics server, the monitored characteristics of the at least one human with the monitored environmental factors proximate the at least one human.

Some embodiments of the method include generating, based on the monitored characteristics of the at least one human or the monitored environmental factors, feedback indicating a schedule for at least one of administering medications, performing rehabilitation activities, meals, and sleep and providing the generated feedback to the at least one human.

In some embodiments, collecting the data includes monitoring motion or behavior of the at least one human using a video camera.

Some embodiments of the method include performing, using the plurality of sensors, concurrent imaging of body organs in at least one human using at least one of structure measurements, electro-neurophysiological measurements, and measurements related to metabolism, neurohumoral physiology, and circulation-related physiology.

Some embodiments of the method include performing, using the ML analysis server, real-time analysis on the data collected during the concurrent imaging of the body organs by the plurality of sensors and generating, using the ML analysis server, feedback for at least one remote user based on the real-time analysis.

Some embodiments of the method include reconfiguring the plurality of sensors based on information received from the at least one remote user in response to the feedback.

Some embodiments of the method include providing at least one of blinded, masked, or anonymized biomarker data and environmental data to a regional cloud.

In some embodiments, an apparatus is provided that includes at least one processor and at least one memory including computer program code. The at least one memory and the computer program code are configured to, with the at least one processor, cause the apparatus at least to perform establishing sensor data provenance to ensure integrity and localization of data collected by a plurality of sensors within the edge cloud network, collecting, using the plurality of sensors, data associated with at least one human, conveying the data to at least one base station within the edge cloud network, storing, at a core network implemented in the edge cloud network, the data collected using the plurality of sensors, and analyzing, using a machine learning (ML) analytics server implemented in the edge cloud network, the data collected using the plurality of sensors.

In some embodiments, a method is provided for implementation in an edge cloud network. The method includes establishing sensor data provenance to ensure integrity and localization of data collected by a plurality of sensors within the edge cloud network and collecting, using the plurality of sensors, data associated with at least one human. The method also includes analyzing, using a machine learning (ML) analytics server implemented in the edge cloud network, the data collected using the plurality of sensors. The method further includes providing personalized medical treatment to the at least one human based on the analysis performed by the ML analytics server.

In some embodiments, providing the personalized medical treatment includes providing non-invasive brain stimulation using transcranial magnetic stimulation (TMS) based on the analysis performed by the ML analytics server.

In some embodiments, providing the personalized medical treatment includes detecting disruptions or alterations of circadian rhythms in the at least one human and providing treatment to the at least one human based on the detected disruptions or altercations of the circadian rhythms.

### BRIEF DESCRIPTION OF THE DRAWINGS

The present disclosure may be better understood, and its numerous features and advantages made apparent to those skilled in the art by referencing the accompanying drawings. The use of the same reference symbols in different drawings indicates similar or identical items.

FIG. 1 is a block diagram of a communication system that supports data provenance, localization, and analysis for personal data collected in a private enterprise network according to some embodiments.

FIG. 2 is a block diagram of a network function virtualization (NFV) architecture according to some embodiments.

FIG. 3 is a block diagram of a communication system that utilizes multiple frequency bands to support communication by a private enterprise network according to some embodiments.

FIG. 4 is a block diagram of a communication system that includes an edge cloud network to support data provenance, localization, and analytics according to some embodiments.

FIG. 5 is a block diagram of a communication system for collecting physiological and environmental data associated with a human according to some embodiments.

FIG. 6 is a block diagram of a communication system for conveying physiological and environmental data associated with a human between sites within a private enterprise network according to some embodiments.

FIG. 7 is a block diagram of a communication system including a regional cloud that is in communication with multiple edge clouds according to some embodiments.

FIG. 8 is a flow diagram of a first portion of a method of configuring and operating a private enterprise network implemented in an edge cloud to support data provenance, isolation, and analytics according to some embodiments.

FIG. 9 is a flow diagram of a second portion of the method of configuring and operating a private enterprise network implemented in an edge cloud to support data provenance, isolation, and analytics according to some embodiments.

FIG. 10 is a flow diagram of a method of defining, tracking, and providing real-time feedback for human physiological biomarkers according to some embodiments.

FIG. 11 includes plots of examples of circadian profiles for different monitored humans according to some embodiments.

### DETAILED DESCRIPTION

The most valuable spectrum available for cellular communication is at frequencies below 6 Gigahertz (GHz) because transmissions at these frequencies do not require a clear line of sight between the transmitter and the receiver. Much of the sub-6-GHz spectrum is already auctioned off as statically licensed spectrum to various mobile network operators (MNOs) that implement cellular communication system such as LTE networks. The 3.1-4.2 GHz spectrum is occupied by incumbents such as Fixed Satellite System (FSS) and federal incumbents such as U.S. government or military entities. For example, the 3550-3700 MHz frequency band (CBRS band) was previously reserved for exclusive use by incumbents including the United States Navy and Fixed Satellite Service (FSS) earth stations. This band of the spectrum is often highly underutilized by incumbents, who nevertheless monopolize access to this band. Consequently, organizations and vertical industries such as package distribution companies, energy producers, ports, mines, hospitals, and universities do not have access to sub-6-GHz spectrum and are therefore unable to establish private enterprise networks to provide cellular service such as LTE.

The Federal Communication Commission (FCC) has begun offering bands of spectrum owned by federal entities for sharing with commercial operations. For example, newly issued FCC rules in 47 Code of Federal Regulations (CFR) Part 96 allows sharing of the 3550-3700 MHz Citizens Broadband Radio Service (CBRS) between incumbents and other operators. The CBRS operates according to a tiered access architecture that distinguishes between incumbents, operators that have received a priority access license (PAL) consistent with 47 CFR § 96.23, et seq., and general authorized access (GAA) operators that are authorized to implement one or more Citizens Broadband radio Service Devices (CBSDs) consistent with 47 CFR § 96.33, et seq. Incumbents, PAL licensees, and GAA operators are required to request access from a spectrum access system (SAS), which allocates frequency bands to the operators, e.g., for CBRS within the 3550-3700 MHz band. The SAS is responsible for managing or controlling different types of CBSDs in the CBRS frequency bands. In current deployments, the CBSD are categorized as:
Category A—CBSDs designed for indoor deployments with a maximum transmission power limit of 30 dBm,
Category B—CBSDs designed for outdoor deployments with a maximum transmission power limit of 47 dBm.
CPE—CBSDs designed for use as customer premises equipment.

The SAS allocates frequency bands to the CBSDs associated with the operators within geographical areas and, in some cases, during scheduled time intervals. The SAS determines whether incumbents are present within corresponding geographical areas using an environmental sensing capability (ESC) that performs incumbent detection, e.g., using radar to detect the presence of a Navy ship in a port.

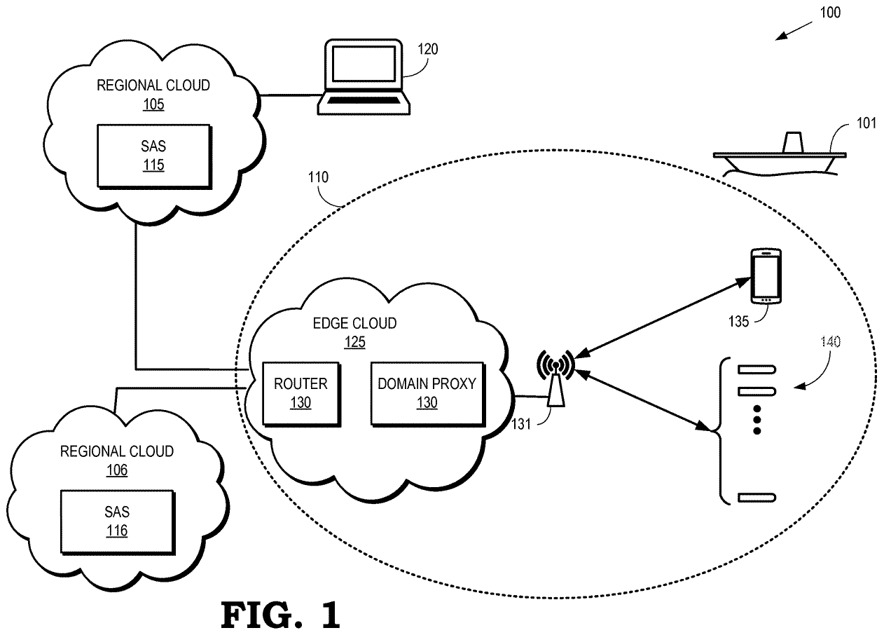

FIGS. 1-10 disclose embodiments of private enterprise networks that provide high bandwidth, reliability, and quality-of-service (QoS), while also providing enhanced privacy and security for user data collected by the enterprise, are implemented in an edge cloud network including one or more base stations that support wireless communication with a plurality of sensors via at least one of licensed spectrum (e.g., in a frequency range less than 6 GHz), a shared Citizens Broadband Radio Service (CBRS) spectrum, unlicensed spectrum, and opportunistically available licensed spectrum through spectrum sub-leasing from companies that hold under-utilized licensed spectrum and are willing to sub-lease portions of the spectrum. The edge cloud network also includes a core network that stores data collected by the enterprise from the plurality of sensors and a machine learning (ML) analytics server to analyse the data collected from the plurality of sensors. Some embodiments of the edge cloud network include one or more access points that are connected to the base stations and provide wireless connectivity to a subset of the plurality of sensors. In some embodiments, a first subset of the plurality of sensors monitors characteristics of one or more humans (e.g., one or more biomarkers or information used to generate the biomarkers) and a second subset of the plurality of sensors monitors environmental factors such as light levels, ambient noise levels, ambient temperatures, and the like. The biomarkers can include counts of eye blinks, an electrocardiogram, pulse plethysmography, body temperature, blood pressure, and the like. The plurality of sensors can also include a video camera to monitor motion or behaviour of one or more humans. Sensor data provenance ensures that the biomarker data streamed to the analytic serve on the edge cloud is not tampered with by a malicious entity or that inconsistent/inaccurate data is not arriving from a faulty or misfunctioning sensor.

Private enterprise networks that implement data provenance, isolation, and analysis are particularly advantageous in medical contexts, at least in part because of the importance of maintaining patient privacy and confidentiality. The consensus of modern medical research is that many diseases are syndromes that include several subgroups and various clinical outcomes that depend on genetic, environmental, and behavioral factors that are unique to each patient. Personalized medicine accounts for the idiosyncrasies of different patients by tailoring both pharmacological and nonpharmacological treatments to the individual, e.g., using patient-specific pharmaceutical prescriptions, recommendations for nutrition, exercise, and sleep hygiene, as well as other non-pharmacological treatments such as neural stimulation. Applying ML techniques to the sensor data acquired for each patient can significantly enhance patient outcomes. For example, ML techniques can be used (during clinical trials or outside the clinical trial phase) to gauge the efficacy of new drugs for the treatment of common diseases such as anxiety disorder, blood pressure, heart and lung diseases, as well as less common diseases such as Alzheimer's disease or Parkinson's disease. For another example, ML techniques can be used to identify times when patients are most (or least) attentive to self-care or treatments are most effective. The timing of rehabilitation or treatment plans can then be tailored to the individual patient's needs. For another example, sensors can be used to gather performance data for humans in different working environments and the performance data can be used to design environments to prevent accidents and modify the performance states of people to enhance the outcomes of work tasks.

Private enterprise networks that implement an ML analytics server can securely gather patient information (such as biomarkers and environmental information) that is stored within the edge cloud, thereby preserving patient privacy. The ML analytics server defines and/or tracks correlated biomarkers to identify conditions such as a positive/negative response to a treatment or cognition impairment. The ML analytics software also correlates the biomarkers with environmental factors such as time of day, light levels, ambient noise levels, ambient temperatures, and the like. In some embodiments, the ML analytics server provides real-time analysis and feedback indicating remedial actions such as recommended meals or medications and schedules for the meals or medications. The real-time analysis can include performing backward and forward loops of information between the private enterprise network and the system of humans and sensors. The feedback loops can be used to control the quality of the data collected by the sensors, to optimize the performance of the system, or to exchange information within the private enterprise network or with an external network. For example, data collected during concurrent imaging of body organs using structure measurements, electro-neurophysiological measurements, and measurements related to metabolism, neurohumoral physiology, and circulation-related physiology can be conveyed to a core network in the private enterprise network. A remote user can then modify the parameters that configure one or more of the imaging sensors based on the received images.

A regional cloud provides cloud-based support for the private enterprise network using one or more servers that are configured to provide operations and maintenance (O&M) management, a customer portal, network analytics, software management, and central security for the private enterprise network. The regional cloud also includes a spectrum access system (SAS) to allocate frequency bands to operators, e.g., to the private enterprise network for CBRS within the 3550-3700 MHz band. Some embodiments of the regional cloud include another ML analytics server to analyse data received from the private enterprise network (as well as other private enterprise networks or edge cloud associated with the regional cloud) such as blinded, masked, or anonymized biomarker data and environmental data.

FIG. 1 is a block diagram of a communication system 100 that supports data provenance, localization, and analysis for personal data collected in a private enterprise network according to some embodiments. The communication system 100 operates in accordance with the FCC rules set forth in 47 Code of Federal Regulations (CFR) Part 96, which allows sharing of the 3550-3700 MHz Citizens Broadband Radio Service (CBRS) between incumbents and other operators. However, some embodiments of the communication system 100 operate in accordance with other rules, standards, or protocols that support sharing of a frequency band between incumbents and other devices such that the frequency band is available for exclusive allocation to an incumbent device if the incumbent device is present in a geographic area. In that case, the other devices are required to vacate any portion of the frequency band that overlaps with another portion of the frequency band that is allocated to the incumbent device. For example, if the communication system 100 is deployed (at least in part) proximate a port and a Navy ship such as an aircraft carrier 101 arrives in the port, devices in a geographic area proximate the port that are providing wireless connectivity in a portion of the frequency band allocated to the aircraft carrier 101 are required to vacate the portion of the frequency band to provide the aircraft carrier 101 with exclusive access to the frequency band within the geographic area. The communication system 100 also supports communication in licensed frequency bands (e.g., according to Long Term Evolution (LTE) standards, Fourth Generation (4G) standards, and Fifth Generation (5G) standards) and unlicensed frequency bands (e.g. according to Bluetooth standards, Wi-Fi standards, or other standards defined by the IEEE).

The communication system 100 includes a regional cloud 105 that provides cloud-based support for a private enterprise network 110. Some embodiments of the regional cloud 105 include one or more servers that are configured to provide operations and maintenance (O&M) management, a customer portal, network analytics, software management, and central security for the private enterprise network 110. The regional cloud 105 also includes an SAS instance 115 to allocate frequency bands to operators, e.g., to the private enterprise network 110 for CBRS within the 3550-3700 MHz band. The communication system 100 also includes another regional cloud 106 that includes an SAS instance 116. In the illustrated embodiment, the regional clouds 105, 106 are located at different geographic locations and are therefore used to provide geo-redundancy. For example, the SAS instance 115 can be selected as a primary SAS and the SAS instance 116 can be selected as a secondary, geo-redundant SAS. The SASs 115, 116 communicate with each other over an SAS-SAS interfaces (not shown in FIG. 1 in the interest of clarity). If additional SAS instances are present in the communication system 100, the SAS instances communicate with each other over corresponding SAS-SAS interfaces. The SASs 115, 116 can serve multiple private enterprise networks, although a single private enterprise network 110 is shown in FIG. 1 in the interest of clarity.

The regional clouds 105, 106 are configured via user interface portals to one or more external computers 120, only one of which is shown in FIG. 1 in the interest of clarity. For example, the external computer 120 can provide a customer user interface portal for service management, a digital automation cloud management user interface portal, and an SAS user interface portal that is used to configure the SASs 115, 116.

The private enterprise network 110 includes an edge cloud 125 that communicates with the regional clouds 105, 106 to support a plug-and-play deployment of the private enterprise network 110. Some embodiments of the edge cloud 125 support auto configuration and self-service, industrial protocols, local connectivity with low latency, cellular (4G LTE or 5G)-based communication and local security, high availability, and other optional applications for the private enterprise network 110. Some embodiments of the edge cloud 125 implement an edge router 128 that facilitates communication with the regional clouds 105, 106. For example, the edge router 128 can download software (or other information) that is used to configure entities in the edge cloud 125 to run as micro-services on the edge cloud 125 including a cellular evolved packet core (EPC), as discussed herein.

In the illustrated embodiment, the edge cloud 125 implements a domain proxy 130 that provides managed access and policy control to a set of CBSDs 131 (only one shown in FIG. 1 in the interest of clarity) that are implemented using base stations, base station routers, mini-macrocells, microcells, indoor/outdoor picocells, femtocells, or other wireless devices or wireless access devices. As used herein, the term “base station” refers to any device that provides wireless connectivity in the private enterprise network 110. Some embodiments of the base station operate as a CBSD, e.g., as either category A CBSD (Indoor), Category B CBSD (outdoor), or customer premises equipment (CPE). The CBSDs 131 are therefore referred to herein as the base stations 131. Some embodiments of the domain proxy 130 are implemented in one of the regional clouds 105, 106.

The domain proxy 130 mediates between the SASs 115, 116 and the base stations 131. In order to utilize the shared spectrum, the base stations 131 transmit requests towards one of the SASs 115, 116 to request allocation of a portion of a frequency band. The other one of the SASs 115, 116 is used as a secondary SAS in case of a failure associated with the primary SAS. The requests include information identifying the portion of the frequency band such as one or more channels, a geographic area corresponding to a coverage area of the requesting base station, and, in some cases, a time interval that indicates when the requested portion of the frequency band is to be used for communication. In the illustrated embodiment, the coverage area of the base stations 131 corresponds to the area encompassed by the private enterprise network 110. Some embodiments of the domain proxy 130 reduce the signal load between the domain proxy 130 and the SASs 115, 116 by aggregating requests from multiple base stations 131 into a smaller number of messages that are transmitted from the domain proxy 130 to the SASs 115, 116. The base stations 131 provide wireless connectivity to corresponding user equipment 135, 136, 137 (collectively referred to herein as “the user equipment 135-137”) in response to the SASs 115, 116 allocating portions of the frequency band to the base stations 131.

The requests transmitted by the base stations 131 do not necessarily include the same information. Some embodiments of the requests from the base stations 131 include information indicating different portions of the frequency band, different geographic areas, or different time intervals. For example, the base stations 131 request portions of the frequency band for use in different time intervals if the private enterprise network 110 is deployed in a mall or shopping center and the base stations 131 are used to provide wireless connectivity within different stores that have different operating hours. The domain proxy 130 therefore manages the base stations 131 using separate (and potentially different) policies on a per-CBSD basis. In some embodiments, the domain proxy 130 accesses the policies for the base stations 131 in response to receiving a request from one of the base stations 131. The domain proxy 130 determines whether the requesting base station from which the request is received is permitted to access the SAS instance 115 based on the policy, e.g., by comparing information in the policy to information in one or more mandatory fields of the request. The domain proxy 130 selectively provides the requests to the SASs 115, 116 depending on whether the requesting base station is permitted to access the SASs 115, 116. If so, the request is transmitted to the SASs 115, 116 or aggregated with other requests for transmission to the SASs 115, 116. Otherwise, the request is rejected.

The base stations 131 support wireless communication with one or more sensors 140. As discussed herein, the sensors 140 include sensors that monitored characteristics of one or more humans, sensors that monitor the environment proximate the one or more humans, cameras that record the motion or behavior of the one or more humans, and the like. The edge cloud network 125 also includes a cellular technology EPC core network (not shown in FIG. 1 in the interest of clarity) that stores data collected using the sensors 140. The edge cloud network 125 further includes a machine learning (ML) analytics server (not shown in FIG. 1 in the interest of clarity) to analyze the data collected using the sensors 140. The edge cloud network 125 implements sensor data provenance to ensure integrity and localization of the data within the edge cloud network 125.

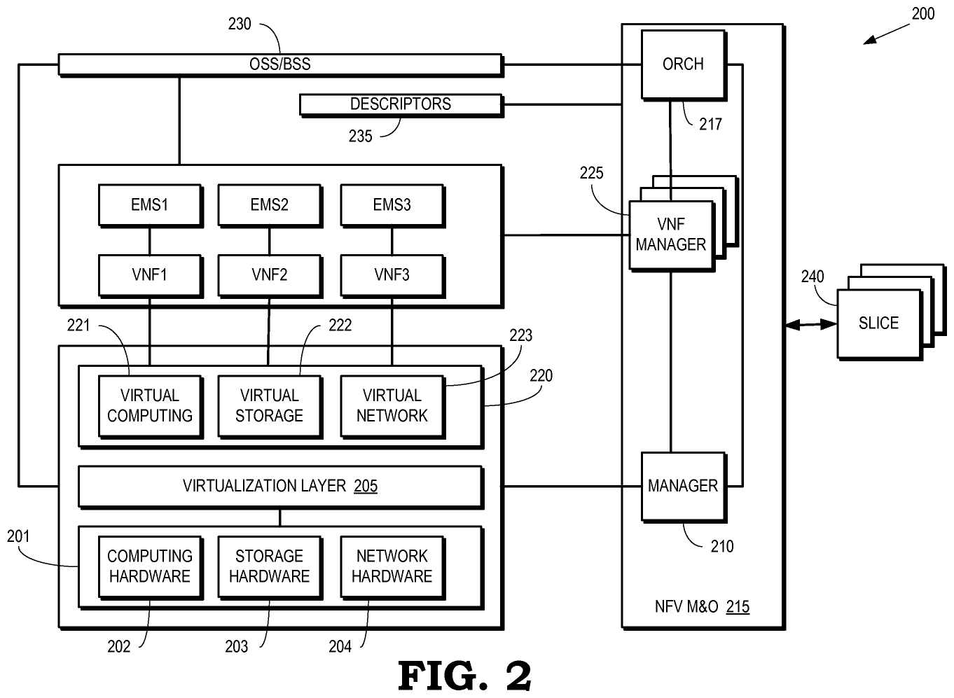

FIG. 2 is a block diagram of a network function virtualization (NFV) architecture 200 according to some embodiments. The NFV architecture 200 is used to implement some embodiments of the communication system 100 shown in FIG. 1. The NFV architecture 200 includes hardware resources 201 including computing hardware 202 such as one or more processors or other processing units, storage hardware 203 such as one or more memories, and network hardware 204 such as one or more transmitters, receivers, or transceivers. A virtualization layer 205 provides an abstract representation of the hardware resources 201. The abstract representation supported by the virtualization layer 205 can be managed using a virtualized infrastructure manager 210, which is part of the NFV management and orchestration (M&O) module 215. Some embodiments of the virtualized infrastructure manager 210 are configured to collect and forward performance measurements and events that may occur in the NFV architecture 200. For example, performance measurements may be forwarded to an orchestrator (ORCH) 217 implemented in the NFV M&O 215. The hardware resources 201 and the virtualization layer 205 may be used to implement virtual resources 220 including virtual computing 221, virtual storage 222, and virtual networking 223.

Virtual networking functions (VNF1, VNF2, VNF3) run over the NFV infrastructure (e.g., the hardware resources 201) and utilize the virtual resources 220. For example, the virtual networking functions (VNF1, VNF2, VNF3) are implemented using virtual machines supported by the virtual computing resources 221, virtual memory supported by the virtual storage resources 222, or virtual networks supported by the virtual network resources 223. Element management systems (EMS1, EMS2, EMS3) are responsible for managing the virtual networking functions (VNF1, VNF2, VNF3). For example, the element management systems (EMS1, EMS2, EMS3) may be responsible for fault and performance management.

In some embodiments, each of the virtual networking functions (VNF1, VNF2, VNF3) is controlled by a corresponding VNF manager 225 that exchanges information and coordinates actions with the virtualized infrastructure manager 210 or the orchestrator 217.

The NFV architecture 200 may include an operation support system (OSS)/business support system (BSS) 230. The OSS/BSS 230 deals with network management including fault management using the OSS functionality. The OSS/BSS 230 also deals with customer and product management using the BSS functionality. Some embodiments of the NFV architecture 200 use a set of descriptors 235 for storing descriptions of services, virtual network functions, or infrastructure supported by the NFV architecture 200. Information in the descriptors 235 may be updated or modified by the NFV M&O 215.

The NFV architecture 200 can be used to implement network slices 240 that provide user plane or control plane functions. A network slice 240 is a complete logical network that provides communication services and network capabilities, which can vary from slice to slice. User equipment can concurrently access multiple network slices 240. Some embodiments of user equipment provide Network Slice Selection Assistance Information (NSSAI) parameters to the network to assist in selection of a slice instance for the user equipment. A single NSSAI may lead to the selection of several network slices 240. The NFV architecture 200 can also use device capabilities, subscription information and local operator policies to do the selection. An NSSAI is a collection of smaller components, Single-NSSAIs (S-NSSAI), which each include a Slice Service Type (SST) and possibly a Slice Differentiator (SD). Slice service type refers to an expected network behavior in terms of features and services (e.g., specialized for broadband or massive IoT), while the slice differentiator can help selecting among several network slice instances of the same type, e.g. to isolate traffic related to different services into different network slices 240.

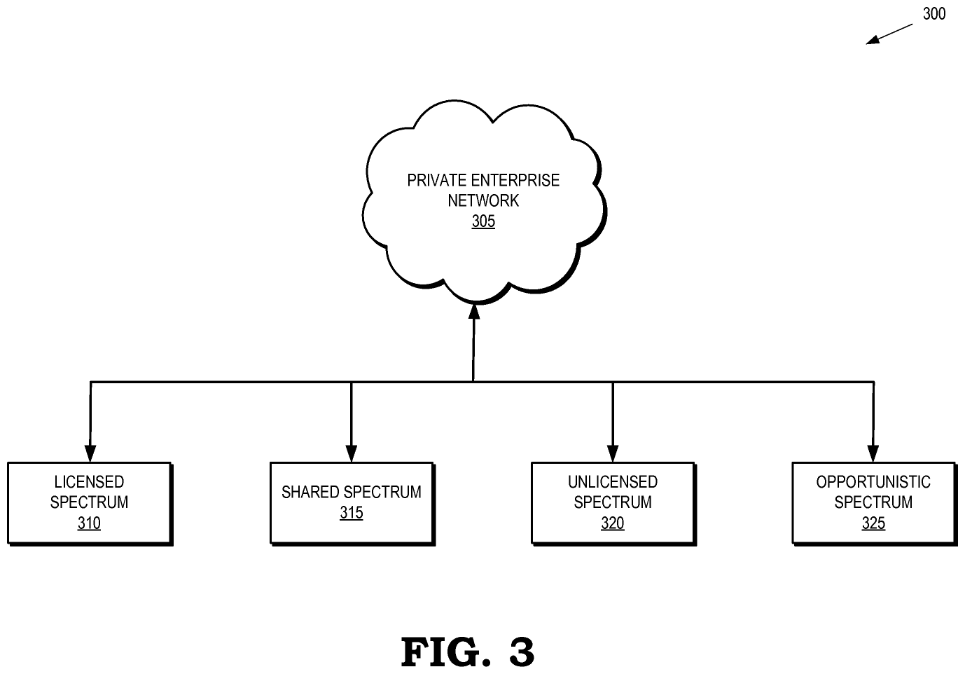

FIG. 3 is a block diagram of a communication system 300 that utilizes multiple frequency bands to support communication by a private enterprise network 305 according to some embodiments. The private enterprise network 305 is used to implement some embodiments of the private enterprise network 110 shown in FIG. 1. Some embodiments of the NFV architecture 200 shown in FIG. 2 are used to implement the private enterprise network 305.

The private enterprise network 305 includes one or more base stations (not shown in FIG. 3 in the interest of clarity) that support wireless connectivity in a set of frequency bands. In the illustrated embodiment, the frequency bands include licensed spectrum 310, a shared spectrum 315 such as a shared Citizens Broadband Radio Service (CBRS) spectrum, an unlicensed spectrum 320, and an opportunistically available licensed spectrum 325. The licensed spectrum 310 is available in a frequency range less than 6 GHz. The shared spectrum 315 can allocate up to 150 MHz within the 3.5 GHz CBRS spectrum. The unlicensed spectrum 320 is available at 5 GHz. The opportunistically available licensed spectrum 325 is made available to the private enterprise network 305 through spectrum sub-leasing from companies that hold under-utilized licensed spectrum and are willing to sub-lease portions of the spectrum to the owner or operator of the private enterprise network 305.

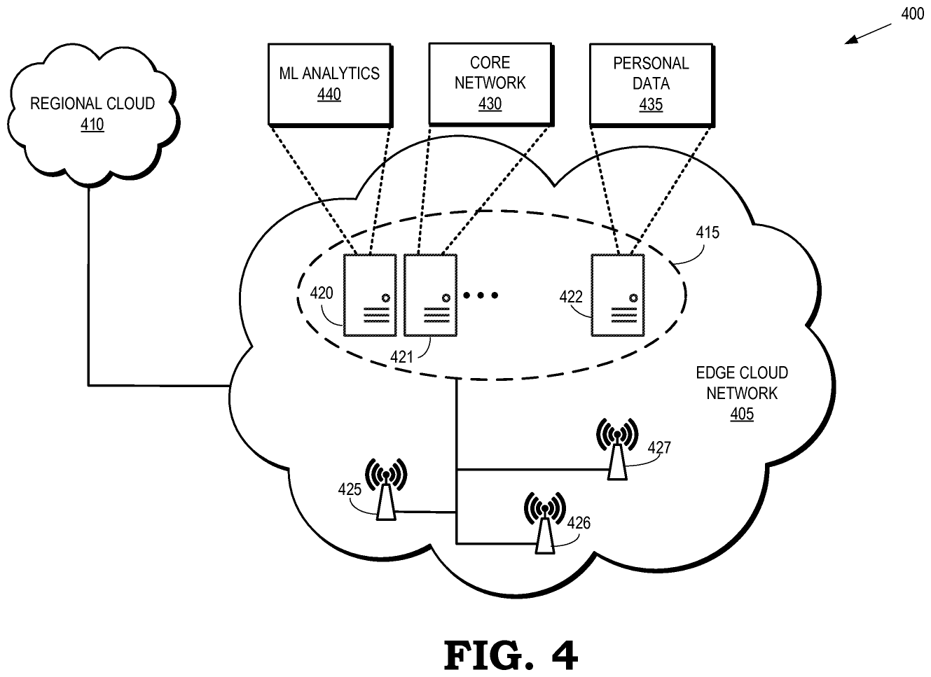

FIG. 4 is a block diagram of a communication system 400 that includes an edge cloud network 405 to support data provenance, localization, and analytics according to some embodiments. The edge cloud network 405 is used to implement some embodiments of the edge cloud 125 shown in FIG. 1. Some embodiments of the NFV architecture 200 shown in FIG. 2 are used to implement the edge cloud network 405. The edge cloud network 405 is connected to one or more regional clouds 410 such as the regional clouds 105, 106 shown in FIG. 1.

The edge cloud network 405 includes a set 415 of servers 420, 421, 422 (collectively referred to herein as “the servers 420-422”) that provide various edge cloud services. The set 415 of servers 420-422 supports wireless connectivity via one or more base stations 425, 426, 427, which are collectively referred to herein as “the base stations 425-427.” Although three servers 420-422 and three base stations 425-427 are shown in FIG. 4, some embodiments include more or fewer servers or base stations. In some embodiments, a private enterprise network is instantiated on one or more of the servers 420-422 and provides wireless connectivity via one or more of the base stations 425-427. For example, the private enterprise network 110 shown in FIG. 1 or the private enterprise network 305 shown in FIG. 3 can be instantiated on the edge cloud network 405. The edge cloud network 405 also includes a core network 430 that is instantiated in one or more of the servers 420-422. In the illustrated embodiment, the core network 430 is downloaded from the regional cloud 410 and installed in the server 421. For example, the core network 430 can be downloaded and installed in response to instantiating a private enterprise network that utilizes the servers 420-422 and the base stations 425-427.

In some embodiments, the edge cloud network 405 collects data associated with humans such as personal medical data and environmental data. As discussed herein, the personal data is collected using a variety of sensors that communicate with the edge cloud network 405 via one or more of the base stations 425-427. Since the core network 430 is installed in the edge cloud network 405, it is not necessary to transmit this data outside of the edge cloud network 405, e.g., to the regional cloud 410. Instead, the personal data 435 is stored in the server 422 (or another one or more of the servers 420-422). Thus, data localization for the personal data 435 is ensured. Furthermore, as discussed herein, trusted data provenance is also ensured for the personal data 435.

An ML analytics module 440 is instantiated in the server 420 (or another one or more of the servers 420-422). The ML analytics module 440 accesses the personal data 435 and performs various analytics and tracking functions. Some embodiments of the ML analytics module 440 identify correlations between biomarkers in the personal data 435 and track the correlated biomarkers in the personal data 435. Tracking of the correlated biomarkers can be performed in real time. As discussed herein, the personal data 435 includes environmental data representing environmental factors proximate the monitor humans. The ML analytics module 440 can therefore correlate the monitored characteristics or biomarkers associated with the human with the monitored environmental factors proximate the human. The ML analytics module 440 also generates feedback based on the monitored characteristics, biomarkers, and environmental factors. In some embodiments, the feedback is provided to the human (or a medical practitioner providing care to the human) and includes schedules for administering medications, calibrating parameters of a treatment, performing rehabilitation activities, eating meals, sleeping, and other activities.

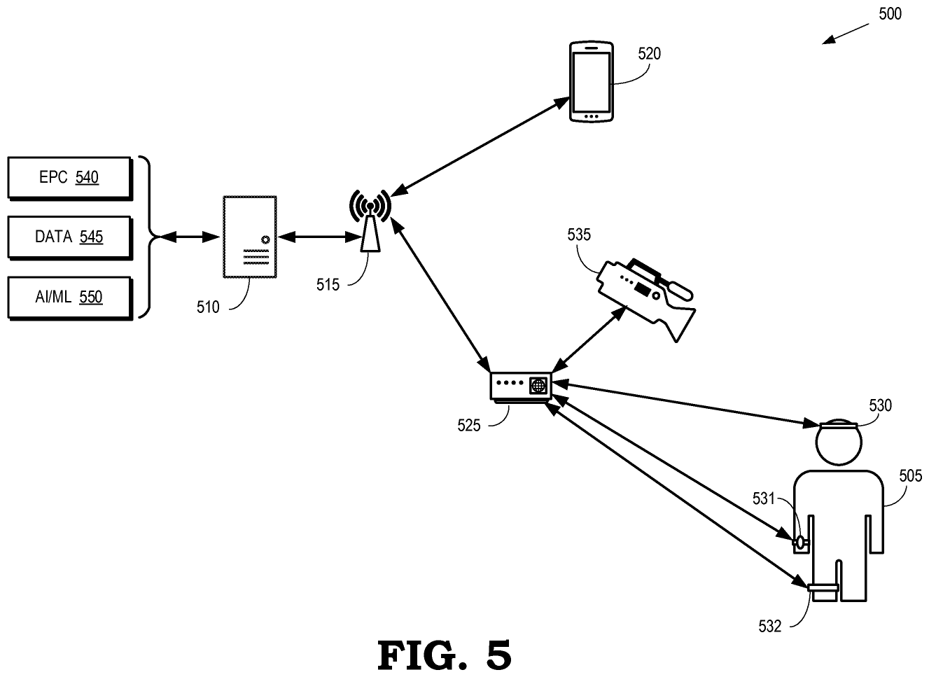

FIG. 5 is a block diagram of a communication system 500 for collecting physiological and environmental data associated with a human 505 according to some embodiments. The communication system corresponds to some embodiments of the communication system 100 shown in FIG. 1 and the communication system 400 shown in FIG. 4. Some embodiments of the NFV architecture 200 shown in FIG. 2 are used to implement the communication system 500. The communication system 500 includes one or more servers 510 that are connected to one or more base stations 515 for providing wireless connectivity to user equipment 520. Although the user equipment 520 shown in FIG. 5 is represented as a smart phone, other types of user equipment 520 including tablets, personal computers, laptops, and the like are implemented in other embodiments. In the illustrated embodiment, the base stations 515 also provide wireless connectivity to one or more access points 525 that also provide wireless connectivity in the same or different frequency bands as the base stations 515. For example, the base stations 515 can operate according to LTE or 5G and the access points 525 can operate according to Bluetooth, Wi-Fi, LTE, 5G, or other standards or protocols.

The communication system 500 includes multiple sensors 530, 531, 532 (collectively referred to herein as “the sensors 530-532”) that collect physiological data associated with the human 505. In the illustrated embodiment, the sensors 530-532 are worn at various locations on the body of the human 505 and collect different types of data. Examples of the physiological data collected by the sensors 530-552 include, but are not limited to, counts of eye blinks, an electrocardiogram, pulse plethysmography, body temperature, pulse rate, and blood pressure. In some embodiments, a subset of the sensors is not worn by the patient. For example, smart space sensors may be able to detect and report human physiological data for one or more humans in the vicinity of the smart space sensor. The communication system 500 also includes one or environmental sensors represented in FIG. 5 by the camera 535. The environmental sensors 535 collect environmental data associated with (or proximate to) the human 505. Examples of the environmental data associated with the human 505 include, but are not limited to, a light level, an ambient noise level, and an ambient temperature. A high accuracy indoor positioning system (HAIP) sensor allows the system to provide a data stream of the patient physical location in space so that the analytics server on the edge cloud may generate highly correlated (space and time) biomarkers.

The server 510 and the sensors 530-532, 535 establish a trusted data provenance relationship to verify the integrity of the data received from the sensors 530-532, 535. In some embodiments, the server implements an attestation server that monitors trustworthiness of the entities in the communication system including monitoring trustworthiness of hardware, software, and/or firmware. The attestation procedure to establish and maintain the trusted data provenance relationship is based on measurements such as reference quotes that are derived upon installation or configuration of a monitored entity (such as one of the sensors 530-532, 535). The reference quotes are compared to quotes that are subsequently derived by entities in the communication system 500 to verify the trusted data provenance relationship. Examples of quotes include trusted platform module (TPM) quotes. In some embodiments, a data source (such as the server 510 or one of the sensors 530-532, 535) combines or bundles source data and a quote into a source data object and then a digital signature is derived by applying a key that is assigned to the data source. The digital signature enables subsequent verification of authenticity and/or integrity of the source data object that includes the source data and the quote. The source data objects are then exchanged between the entities, which apply corresponding keys to verify authenticity and/or integrity of the received source data object.

Some embodiments of the server 510 instantiate a core network such as an evolved packet core (EPC) 540 that is implemented within a private enterprise network. Personal data 545 including the physiological data and the environment of the data associated with the human 505 is also stored in the server 510. The servers 510 also implement an artificial intelligence (AD/machine learning (ML) module 550 that performs analysis on the personal data 545, as discussed herein. Thus, data localization of the personal data 545 is ensured within the communication system 500.

Some embodiments of the communication system 500 are used to implement personalized medical monitoring and treatment such as transcranial magnetic stimulation (TMS), which is a method of non-invasive brain stimulation that is considered one of the most effective treatments for types of depression that react poorly to anti-depressants. In the illustrated embodiment, the sensor 530 includes a TMS device that is made of one or more copper coils, positioned superficially to a site of interest in the brain, to non-invasively produce a brief magnetic pulse to an estimated depth from the surface of the scalp to produce axonal depolarization in the brain of the human 505. This axonal depolarization activates cortical and subcortical networks with multiple effects.

The communication system 500 allows the TMS treatment of the human 505 to be personalized by obtaining fast, real-time feedback on the effect of the stimulation on the different electrophysiological systems in the body of the human 505. To further fine-tune TMS, the sensors 530-532 and the camera 535 gather information on a wide range of biophysiological systems operating in the human 505. In some cases, substantially continuous monitoring is performed to account for the effects of circadian rhythms, adaptation to internal factors such as prolonged wakefulness, increased sleep pressure, and adaptation to different types of stressful events such as temporary diseases (e.g. infections, trauma). Age of the human 505 also has a profound and complex effect on the different physiological systems. The intra-bodily systems also adapt to external factors (such as light, auditory, visual, temperature stimuli) and the amount of loading resulting from carrying out daily tasks. The interactions between the different systems influence human behavior. Disease-related changes in electrophysiological dynamics in the different biological systems also influence the efficacy and outcomes of TMS treatments.

The sensors 530-532 and the camera 535 gather a wide range of different types of data on human bodily functions. In some embodiments, the data that is gathered includes data on the sleep-wake cycle experienced by the human 505. Nearly all diseases affect the sleep-wake cycle to some extent. Research has shown that disruptions of sleep-wake patterns and daytime vigilance are common in diseases such as Alzheimer's and Parkinson's disease and different mental disorders. Disruptions in the sleep-wake cycle are also common also in post-brain stroke and traumatic brain injuries. Conventional treatments for these diseases do not account for the effect of time of day, sleep-wake cycles affect bio-physiology on different types of treatments. For example, protocols for taking drugs or providing non-medical rehabilitation (such has physiotherapy) do not typically consider a person's level of vigilance or fatigue at a certain time of day. Also, problems with sleep-wake cycle are often missed as possible causes of adverse effects of drug or the root cause to not getting a good enough response with drugs or non-medical rehabilitation. The data 545 gathered by the sensors 530-532, 535 are stored in memory of the server 510.

The AI/ML module 550 performs analysis of the gathered data 545 using ML-based data analytics algorithms to define and track new highly correlated (space, time, and human physiological) biomarkers. The AI/ML module 550 provides real-time biofeedback based on analysis of the gathered data 545. In some embodiments, the biofeedback indicating the dynamics and state of the different biophysiological systems are provided to the person being monitored or to an expert treating the individual. The biofeedback can also control treatments or therapies supplied to the human 505. For example, in TMS, the feedback is used to control an electrical current that is applied to the human 505 via the sensor 530 with different frequencies. The AI/ML module 550 selects the amplitude and the frequencies of the electrical current to repair sleep structure and sleep-wake rhythm. Research has described several types of biofeedback neurostimulation approaches. The AI/ML module 550 therefore fine tunes and individualizes neural stimulation via a feedback loop including the human 505, the sensors 530-532, 535, and the AI/ML module 550. The feedback loop also provides direct information on the effects of neural stimulation on the electrophysiological signal.

Some embodiments of the communication system 500 are used to monitor, diagnose, treat, or evaluate treatments of sleep-wake circadian rhythm clock disruption (SCRD). A circadian clock is present in all living organisms and is the most important internal biorhythm for pacing human biophysiological and cognitive performance. The circadian clock system in humans is regulated by a “major clock” in the brain. The circadian clock system also includes clock genes that are found in all cells and organs of the human body. Mismatches between the rhythms of the circadian clock system and the rhythms of the sleep-wake cycle can result in SCRD in humans. The number of people working at odd hours and against their personal circadian rhythm is increasing and SCRD is becoming epidemic and pandemic. Furthermore, SCRD raises the risk of illnesses including sleep disorders, heart disease, hypertension, stroke, diabetes, chronic and acute infections, autoimmune diseases such as asthma and rheumatoid arthritis, several types of cancer, memory diseases, and psychiatric disorders including anxiety, depression, and manic-depressive psychosis. Chronic stress states triggered by SCRD can also speed aging so that the person suffering SCRD looks and performs like a much older person, e.g., a 45-year-old person can appear and function like a 65-year-old person. Human biorhythms, and therefore SCRD, also affect human responses to different types of treatments including drugs, rehabilitation, recovery from operations, and the like.

In the illustrated embodiment, the sensors 530-532 and 535 include sensors that measure and monitor various physiological bio-signals that are indicative of organ function or other aspects of human physiology that are affected by circadian rhythms. The sensors 530-532 and 535 are implemented as personal wearables, non-contact sensors implemented in smart furniture or in a smart space, or other devices that collect physiologic data from multiple sources (Internet of Things, IoT). Indicators of individual circadian rhythms that are monitored by the sensors 530-532, 535 include, but are not limited to, temporal variations in core body temperature, secretion of melatonin, secretion of cortisol hormones, phasic rhythm of eye blinks, and other measurable biomarkers or metrics of performance associated with the function of physiologic systems including cardiovascular, metabolic, hormonal, and gastrointestinal systems. The data collected by the sensors 530-532, 535 are used to create circadian profiles of bodily functions with an appropriate accuracy. The measurements can be continuous measurements, regular point measurements, or a combination thereof. The sensors 530-532, 535 can include devices with medical grade approval and consumer-oriented trackers.

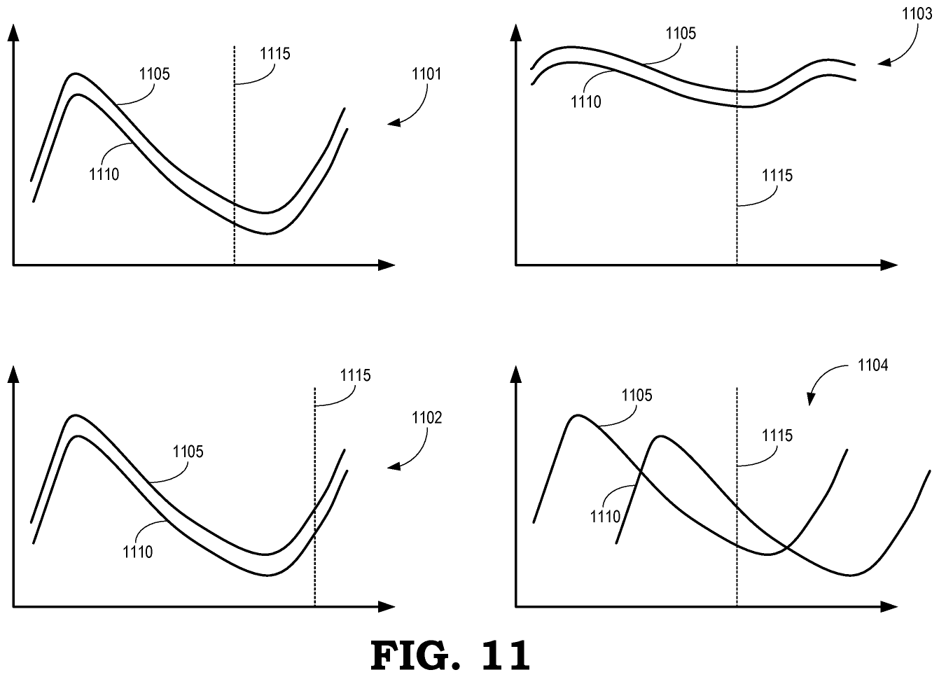

FIG. 11 includes plots 1101, 1102, 1103, 1104 of examples of circadian profiles for different monitored humans according to some embodiments. The plots 1101-1104 include profiles of the monitored humans heart rate 1105 and core body temperature 1110 as a function of time (increasing from left to right) for one day. Midnight is indicated by the dashed line 1115. The plot 1101 shows the heart rate 1105 and core body temperature 1110 of a normal or typical human subject. In some embodiments, the circadian rhythms of other biomarkers are measured such as pulse rate variability, blood pressure, respiratory airflow, skin blood flow, skin elasticity, and the like. The heart rate 1105 and the core body temperature 1110 follow substantially the same circadian profile with a midmorning peak and a valley shortly after midnight. The plot 1102 shows the heart rate 1105 and core body temperature 1110 of a human subject having circadian profiles that are displaced relative to the normal profiles shown in the plot 1101, e.g., the subject of the plot 1102 is experiencing jet lag. The plot 1103 shows the heart rate 1105 and the core body temperature 1110 of a human subject having flattened and elevated circadian profiles relative to the normal profiles shown the plot 1101, e.g., the subject of the plot 1103 is experiencing an acute infection that has changed the circadian profiles. The plot 1104 shows asynchrony between the (normally synchronize) heart rate 1105 and core body temperature 1110 of a human subject. The asynchrony can be caused by disease, a side effect of medication, or other causes.

The communication system 500 detects disruptions or alterations of circadian rhythms in the user 505 based on the biomarkers monitored by the sensors 530-532, 535. Treatments are provided to the user 505 to address the detected disruptions or alterations in their circadian rhythms. In some embodiments, the server 510 stores data 545 representative of the monitored biomarkers for subsequent analysis by the AI/ML module 550. The AI/ML module 550 determines a baseline circadian profile for the user 505, e.g., corresponding to the plot 1101 shown in FIG. 11. The AI/ML module 550 then learns patterns in the fluctuations or variations of the monitored biomarkers that indicate disruptions or alterations in the circadian rhythms of the user 505. Examples of the fluctuations or variations that can be detected by the AI/ML module 550 are shown in the plots 1102-1104 shown in FIG. 11. The server 510 provides feedback based on the monitoring, including feedback provided to the user 505, doctors or medical professionals that are caring for the user 505, medical devices that are providing treatment to the user 505, systems that are assessing the quality of the treatment provided to the user 505, and the like. In some embodiments, the feedback is visualized as a clock table, a set of numerical values, one or more graphs, one or more body maps, and the like.

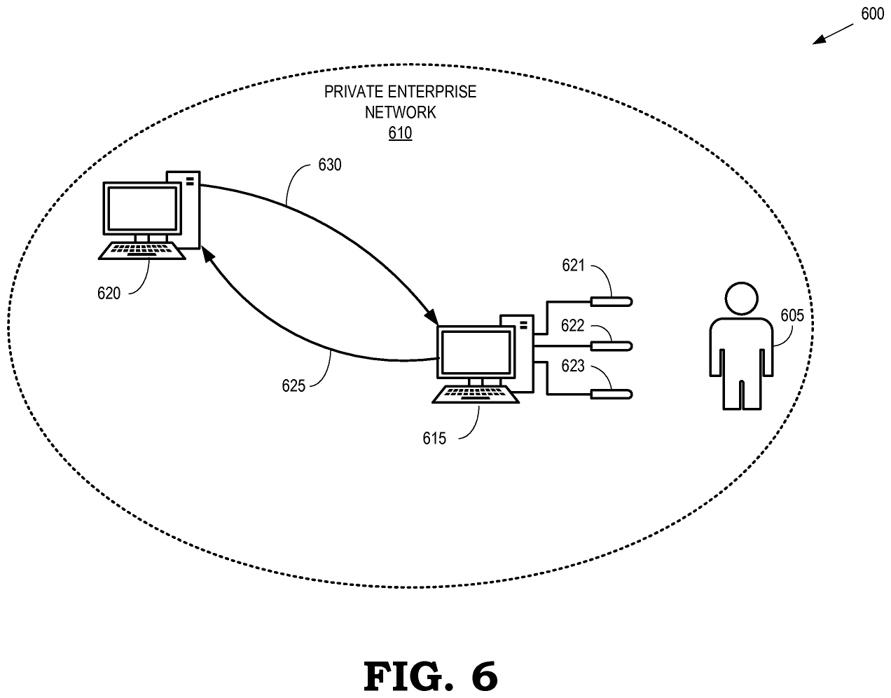

FIG. 6 is a block diagram of a communication system 600 for conveying physiological and environmental data associated with a human 605 between sites within a private enterprise network 610 according to some embodiments. The communication system 600 corresponds to some embodiments of the communication system 100 shown in FIG. 1, the communication system 400 shown in FIG. 4, and the communication system 500 shown in FIG. 5. Some embodiments of the NFV architecture 200 shown in FIG. 2 are used to implement the communication system 600.

The private enterprise network 610 includes computing systems 615, 620 located at different physical locations within the same private enterprise network 610. The computing system 615 is connected to one or more sensors 621, 622, 623 (collectively referred to herein as “the sensors 621-623”) that collect physiological and environmental data associated with the human 605. The computing system 615 has established a trusted data provenance relationship with the sensors 621-623 and the computing system 620. A high bandwidth connection 625, 630 is established between the computing systems 615, 620 and allows the computing system 615 to transmit large volumes of data collected by the sensors 621-623 to the computing system 620. The personal data is stored by the computing system 620, and, in some embodiments, the computing system 620 analyzes the personal data using an ML analytics module, as discussed herein. The computing system 620 uses the high bandwidth connection 625 to transmit feedback to the computing system 615 such as feedback that is used to reconfigure one or more of the sensors 621-623. Backward and forward loops are established using the high bandwidth connections 625, 630 between the computing system 615, 620 can therefore be used to control the quality of the data collected by the sensors 621-623, to optimize performance of the system, and to exchange information inside the edge cloud that implements the private enterprise network 610.

Some embodiments of the private enterprise network 605 implement fusion imaging to combine information indicating structure and functional dynamics of organs in the human 605. The private enterprise network 605 therefore supports remote, real-time monitoring of relationships and interactions between structural elements (e.g., the organs in the human 605) and the sensors 621-623 that sense the performance of a system (e.g., the human 605). For example, the computing systems 615, 620 and the private enterprise network 605 combine the results of imaging different body organs using different techniques and sensors 621-623. Data collected by the sensors 621-623 is streamed from the computing system 615 to the computing system 620 over the high bandwidth connection 625 concurrently with analysis being performed by the computing system 620 and continued scanning of the human 605 by the sensors 621-623. Examples of the types of imaging or scanning performed by the sensors 621-623 include, but are not limited to:
a) structure: CT (computer tomography), CBCT (Cone Beam computer tomography,) and MRI (magnetic resonance imaging),
b) Electro Neurophysiology: EEG (electroencephalography), ECG (electrocardiogram), RIP (respiratory inductance plethysmography),
c) metabolism, neurohumoral and circulation-related physiology: fMRI (functional magnetic resonance imaging), PET (positron emission-tomography), ultrasound, fNIRS (functional near-infrared spectroscopy).

Variable combinations of imaging techniques are performed concurrently by the sensors 621-623. Separate channels of the data stream 625 are used to convey the data collected by the sensors 621-623 to the computing system 620. Some embodiments of the computing system 615 fuse the collected data prior to transmitting the data to the computing system 620. For example, the computing system 615 can be implemented in a hospital, separate laboratory, out-patient setting, or home settings. The fused data is then streamed to the computing system 620 in separate channels of the high bandwidth connection 625 and fused together again at viewing computing system 620. A circular loop (including the streams 625, 630) enables a remote viewer at the computing system 620 to adjust the scanning performed by the sensors 621-623 according to what is seen at computing system 620.

Some embodiments of the sensing system including the sensors 621-623 support different types of sensing data channels, as well as separate bi-or multi-directional channels for audio and video. The private enterprise network 610 can support one-to-one communication between the computing systems 615, 620 or a multichannel system in which fusion data from the computing system 615 is viewed at multiple remote locations including the computing system 620. The additional video and audio streams allow real-time communication both with the human 605 being scanned and multiple viewers including a primary viewer B and secondary viewers C . . . N, which can be added to the system to support real-time communication/discussion of the data being gathered by the sensors 621-623. In some embodiments, the real-time aspects of transferring sensor measurement data (e.g., bio-markers data for the human 605), location information using High Accuracy Indoor positioning capability, and audio/video streams with different priorities in case of network congestion or packet loss, and the need for correctly combining and synchronizing these media and data streams at the receiver end, is provided by using Real-Time Protocol (RTP) to transport these streams. Other specifications related to RTP could be used for indicating the stream prioritization, retransmissions, and session setup.

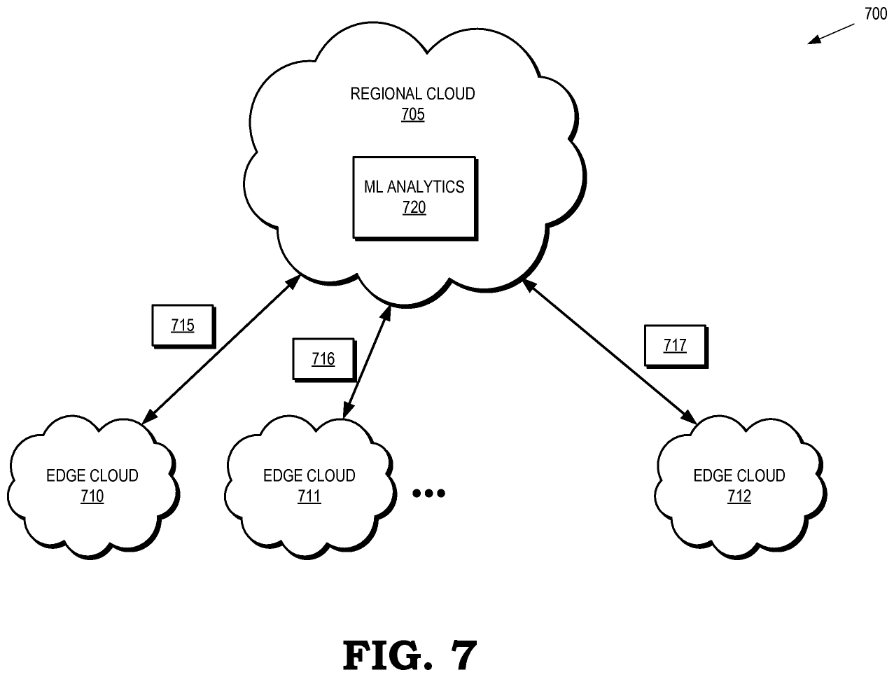

FIG. 7 is a block diagram of a communication system 700 including a regional cloud 705 that is in communication with multiple edge clouds 710, 711, 712 according to some embodiments. The communication system 600 corresponds to some embodiments of the communication system 100 shown in FIG. 1 and the communication system 400 shown in FIG. 4. Some embodiments of the NFV architecture 200 shown in FIG. 2 are used to implement the communication system 700. The edge clouds 710-712 implement corresponding private enterprise networks such as the private enterprise network 305 shown in FIG. 3, the private enterprise network implemented in the edge cloud network 415 shown in FIG. 4, and the private enterprise network 610 shown in FIG. 6.

The private enterprise networks implemented in the edge cloud 710-712 perform data collection operations using sensors that have a trusted data provenance relationship with each other and with other entities in the private enterprise networks, as discussed herein. Core networks are implemented in the private enterprise networks to ensure data isolation for the data collected in the private enterprise networks. In some embodiments, the private enterprise networks implement ML analytics modules to perform analysis on the collected data. The edge clouds 710-712 transmit data 715, 716, 717 (collectively referred to herein as “the data 715-717”) to the regional cloud 705 for further analysis. In some embodiments, the data 715-717 includes blinded, masked, or anonymized data to protect the privacy of the people associated with the data in accordance with patient data privacy regulations. For example, the data 715-717 can include blinded or masked patient biomarker data and analytic results from the ML analytics modules implemented on the edge clouds 710-712. The data 715-717 can also include timestamps to indicate when the data was collected. In some embodiments, common reference clocks in the regional cloud 705 and the edge clouds 710-712 are used to synchronize data collection and analysis. The data 715-717 can also include information indicating relative priorities and can be transmitted using data streams that have different priorities.

The regional cloud 705 implements an ML analytics module 720 for analyzing the data 715-717 received from the edge clouds 710-712. In some embodiments, the ML analytics module 720 performs analysis on masked biomarkers included in the data 715-717. Performing the ML analytics on the regional cloud 705 using data 715-717 acquired from the edge clouds 710-712 allows the ML analytics module 720 to develop broader insights and correlations of human biomarker data over larger data sets, which allows the ML analytics module 720 to draw additional inferences about human performance, cognitive impairments, and the like.

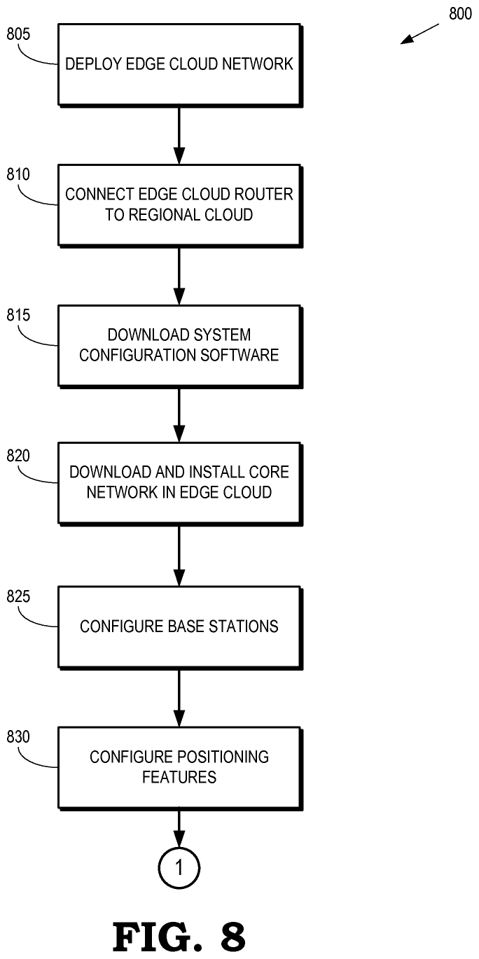

FIG. 8 is a flow diagram of a first portion 800 of a method of configuring and operating a private enterprise network implemented in an edge cloud to support data provenance, isolation, and analytics according to some embodiments. The method is implemented in some embodiments of the communication system 100 shown in FIG. 1, the communication system 400 shown in FIG. 4, the communication system 500 shown in FIG. 5, the communication system 600 shown in FIG. 6, and the communication system 700 shown in FIG. 7.

At block 805, an edge cloud network is deployed, e.g., using some embodiments of the NFV architecture 200 shown in FIG. 2. At block 810, an edge cloud router in the edge cloud network connects to a regional cloud and downloads (at block 815) system configuration software for the edge cloud network. At block 820, a core network is downloaded and installed in the edge cloud network. At block 825, the base stations in the edge cloud are configured. In some embodiments, positioning features associated with sensors or humans in the edge cloud are configured, e.g., by configuring high accuracy indoor positioning features on the edge cloud. The method then flows to the node 1.

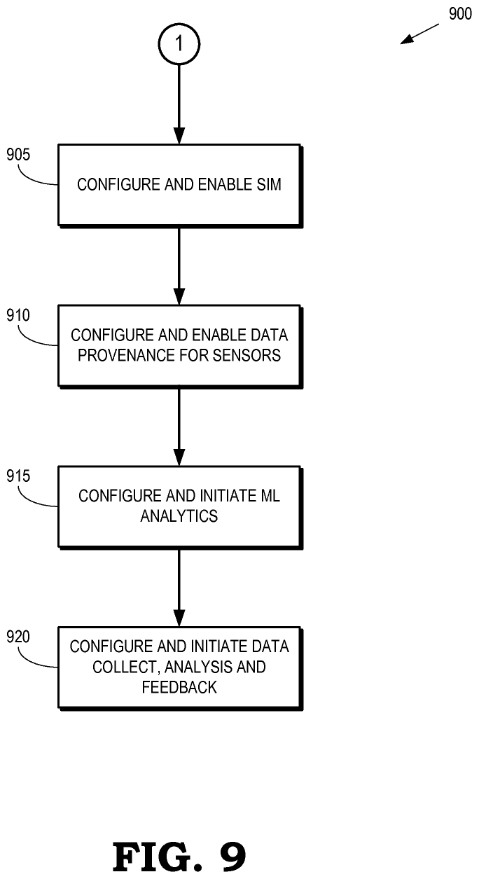

FIG. 9 is a flow diagram of a second portion 900 of the method of configuring and operating a private enterprise network implemented in an edge cloud to support data provenance, isolation, and analytics according to some embodiments. As discussed herein, the method is implemented in some embodiments of the communication system 100 shown in FIG. 1, the communication system 400 shown in FIG. 4, the communication system 500 shown in FIG. 5, the communication system 600 shown in FIG. 6, and the communication system 700 shown in FIG. 7.

The second portion 900 of the method begins at the node 1. At block 905, one or more SIM cards installed on customer premises equipment (CPE) or user equipment (UE) that utilize the private enterprise network are configured and enabled. At block 910, trusted data provenance is configured and enabled for the sensors that are implemented in the private enterprise network, e.g., Internet of Medical Things (IoMT) sensors. At block 915, one or more ML based data analytics modules are configured and initiated, e.g., as micro services deployed on an edge router. The ML based data analytics modules begin generating and tracking new or existing biomarkers in data collected by the sensors. At block 920, data collection and distribution are configured and enabled in the private enterprise network. In some embodiments, real-time analysis and feedback are also configured and enabled in the private enterprise network.

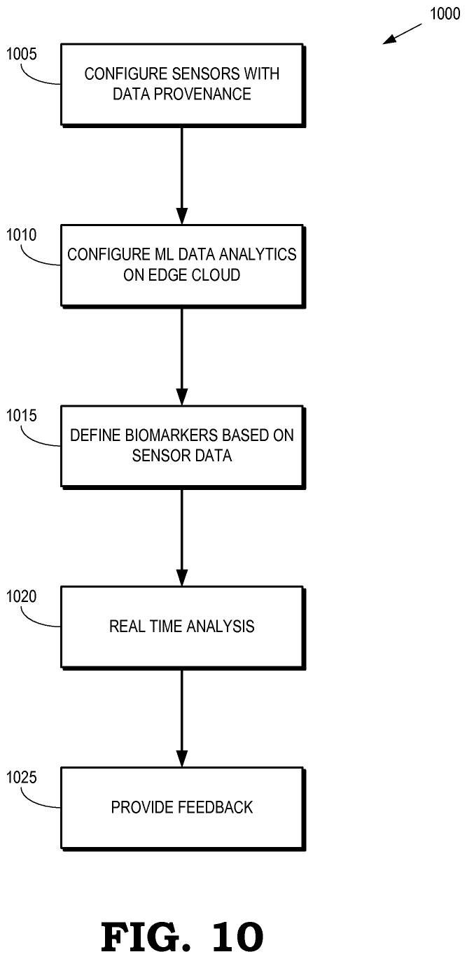

FIG. 10 is a flow diagram of a method 1000 of defining, tracking, and providing real-time feedback for human physiological biomarkers according to some embodiments. As discussed herein, the method 1000 is implemented in some embodiments of the communication system 100 shown in FIG. 1, the communication system 400 shown in FIG. 4, the communication system 500 shown in FIG. 5, the communication system 600 shown in FIG. 6, and the communication system 700 shown in FIG. 7.

At block 1005, sensors in a private enterprise network are configured to support trusted data provenance with other entities in the private enterprise network. At block 1010, an ML data analytics module is configured and deployed in the private enterprise network on the edge cloud. The ML data analytics module can be deployed in a micro service.

At block 1015, the ML data analytics module accesses data acquired by the sensors in the private enterprise network and defines one or more biomarkers based on the sensor data. Examples of the biomarkers defined by the ML data analytics module include, but are not limited to, sleep quality, autonomic nervous system (ANS) physiology, central nervous system (CNS) physiology, and attention to cues provided to the patient. The biomarkers are used to detect characteristics of the human such as cognitive impairment, behavioral changes, and the like. In some embodiments, the ML data analytics module defines the biomarkers based, at least in part, on environmental factors included in the data such as ambient sound, light, and temperature.

At block 1020, the ML data analytics module performs real-time analysis on data acquired by the sensors. At block 1025, the ML data analytics module provides real-time feedback based on the analysis, e.g., to the human subject, doctors, researchers, and the like. In some cases, the real-time feedback is used to determine the timing of actions performed by the human subject including appropriate times to take medications to avoid adverse events that may result from taking the medication at the wrong time. The real-time feedback can also be used to schedule other actions such as performing rehabilitation activities, eating meals, sleeping, and other activities.

In some embodiments, certain aspects of the techniques described above may implemented by one or more processors of a processing system executing software. The software comprises one or more sets of executable instructions stored or otherwise tangibly embodied on a non-transitory computer readable storage medium. The software can include the instructions and certain data that, when executed by the one or more processors, manipulate the one or more processors to perform one or more aspects of the techniques described above. The non-transitory computer readable storage medium can include, for example, a magnetic or optical disk storage device, solid state storage devices such as Flash memory, a cache, random access memory (RAM) or other non-volatile memory device or devices, and the like. The executable instructions stored on the non-transitory computer readable storage medium may be in source code, assembly language code, object code, or other instruction format that is interpreted or otherwise executable by one or more processors.

A computer readable storage medium may include any storage medium, or combination of storage media, accessible by a computer system during use to provide instructions and/or data to the computer system. Such storage media can include, but is not limited to, optical media (e.g., compact disc (CD), digital versatile disc (DVD), Blu-Ray disc), magnetic media (e.g., floppy disc, magnetic tape, or magnetic hard drive), volatile memory (e.g., random access memory (RAM) or cache), non-volatile memory (e.g., read-only memory (ROM) or Flash memory), or microelectromechanical systems (MEMS)-based storage media. The computer readable storage medium may be embedded in the computing system (e.g., system RAM or ROM), fixedly attached to the computing system (e.g., a magnetic hard drive), removably attached to the computing system (e.g., an optical disc or Universal Serial Bus (USB)-based Flash memory), or coupled to the computer system via a wired or wireless network (e.g., network accessible storage (NAS)).

As used herein, the term “circuitry” may refer to one or more or all of the following:
(a) hardware-only circuit implementations (such as implementations and only analog and/or digital circuitry) and
(b) combinations of hardware circuits and software, such as (as applicable):
   (i) a combination of analog and/or digital hardware circuit(s) with software/firmware and
   (ii) any portions of a hardware processor(s) with software (including digital signal processor(s), software, and memory(ies) that work together to cause an apparatus, such as a mobile phone or server, to perform various functions) and
(c) hardware circuit(s) and/or processor(s), such as a microprocessor(s) or a portion of a microprocessor(s), that requires software (e.g., firmware) for operation, but the software may not be present when it is not needed for operation.

This definition of circuitry applies to all uses of this term in this application, including in any claims. As a further example, as used in this application, the term circuitry also covers an implementation of merely a hardware circuit or processor (or multiple processors) or portion of a hardware circuit or processor and its (or their) accompanying software and/or firmware.

The term circuitry also covers, for example and if applicable to the particular claim element, a baseband integrated circuit or processor integrated circuit for a mobile device or a similar integrated circuit in a server, a cellular network device, or other computing or network device.

Note that not all of the activities or elements described above in the general description are required, that a portion of a specific activity or device may not be required, and that one or more further activities may be performed, or elements included, in addition to those described. Still further, the order in which activities are listed are not necessarily the order in which they are performed. Also, the concepts have been described with reference to specific embodiments. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of the present disclosure.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims. Moreover, the particular embodiments disclosed above are illustrative only, as the disclosed subject matter may be modified and practiced in different but equivalent manners apparent to those skilled in the art having the benefit of the teachings herein. No limitations are intended to the details of construction or design herein shown, other than as described in the claims below. It is therefore evident that the particular embodiments disclosed above may be altered or modified and all such variations are considered within the scope of the disclosed subject matter. Accordingly, the protection sought herein is as set forth in the claims below.

1-30. (canceled)

31. An edge cloud network comprising:
at least one base station that supports wireless communication with a plurality of sensors;
a core network that stores data collected using the plurality of sensors; and
a machine learning, ML, analytics server configured to analyze the data collected using the plurality of sensors, wherein the edge cloud network implements sensor data provenance to ensure integrity and localization of the data within the edge cloud network.

32. The edge cloud network of claim 31, wherein the plurality of sensors comprises:
a first subset configured to monitor characteristics of at least one human; and
a second subset configured to monitor environmental factors proximate the at least one human

33. The edge cloud network of claim 31, wherein the first subset of the plurality of sensors is configured to monitor biomarkers comprising at least one of counts of eye blinks, an electrocardiogram, pulse plethysmography, body temperature, and blood pressure, and wherein the second subset of the plurality of sensors is configured to monitor at least one of a light level, an ambient noise level, and an ambient temperature.

34. The edge cloud network of claim 31, wherein the ML analytics server is configured to identify correlations between the biomarkers and track the correlated biomarkers.

35. The edge cloud network of claim 31, wherein the ML analytics server is configured to correlate the monitored characteristics of the at least one human with the monitored environmental factors proximate the at least one human.

36. The edge cloud network of claim 31, wherein the ML analytics server is configured to generate, based on the monitored characteristics of the at least one human or the monitored environmental factors, feedback indicating a schedule for at least one of administering medications, performing rehabilitation activities, meals, and sleep, and wherein the generated feedback is provided to the at least one human.

37. The edge cloud network of claim 31, wherein the plurality of sensors is configured to perform concurrent imaging of body organs in at least one human using at least one of structure measurements, electro-neurophysiological measurements, and measurements related to metabolism, neurohumoral physiology, and circulation-related physiology.

38. The edge cloud network of claim 31, wherein the ML analysis server is configured to perform real-time analysis on the data collected during the concurrent imaging of the body organs by the plurality of sensors, and wherein the ML analysis server is configured to generate feedback for at least one remote user based on the real-time analysis.

39. The edge cloud network of claim 31, wherein the edge cloud network is configured to provide at least one of blinded, masked, or anonymized biomarker data and environmental data to a regional cloud.

40. A method for implementation in an edge cloud network, the method comprising:
establishing sensor data provenance to ensure integrity and localization of data collected by a plurality of sensors within the edge cloud network;
collecting, using the plurality of sensors, data associated with at least one human;
conveying the data to at least one base station within the edge cloud network;
storing, at a core network implemented in the edge cloud network, the data collected using the plurality of sensors; and
analyzing, using a machine learning, ML, analytics server implemented in the edge cloud network, the data collected using the plurality of sensors.

41. The method of claim 40, further comprising:
monitoring, using a first subset of the plurality of sensors, characteristics the at least one human; and
monitoring, using a second subset of the plurality of sensors, environmental factors proximate the at least one human.

42. The method of claim 40, wherein monitoring the characteristics of the at least one human comprises monitoring biomarkers comprising at least one of counts of eye blinks, an electrocardiogram, pulse plethysmography, body temperature, and blood pressure, and wherein monitoring the environmental factors comprises monitoring at least one of a light level, an ambient noise level, and an ambient temperature.

43. The method of claim 40, further comprising:
identifying, using the ML analytics server, correlations between the biomarkers; and
tracking, using the ML analytics server, the correlated biomarkers.

44. The method of claim 40, further comprising:
correlating, using the ML analytics server, the monitored characteristics of the at least one human with the monitored environmental factors proximate the at least one human

45. The method of claim 40, further comprising:
generating, based on the monitored characteristics of the at least one human or the monitored environmental factors, feedback indicating a schedule for at least one of administering medications, performing rehabilitation activities, meals, and sleep; and
providing the generated feedback to the at least one human.

46. The method of claim 40, further comprising:
performing, using the plurality of sensors, concurrent imaging of body organs in at least one human using at least one of structure measurements, electro-neurophysiological measurements, and measurements related to metabolism, neurohumoral physiology, and circulation-related physiology.

47. The method of claim 40, further comprising:
performing, using the ML analysis server, real-time analysis on the data collected during the concurrent imaging of the body organs by the plurality of sensors; and
generating, using the ML analysis server, feedback for at least one remote user based on the real-time analysis.

48. The method of claim 40, further comprising:
reconfiguring the plurality of sensors based on information received from the at least one remote user in response to the feedback.

49. The method of claim 40, further comprising:
providing at least one of blinded, masked, or anonymized biomarker data and environmental data to a regional cloud.

50. A computer readable medium comprising program instructions for causing an apparatus to perform at least the following:
establishing sensor data provenance to ensure integrity and localization of data collected by a plurality of sensors within the edge cloud network;
collecting, using the plurality of sensors, data associated with at least one human;
conveying the data to at least one base station within the edge cloud network;
storing, at a core network implemented in the edge cloud network, the data collected using the plurality of sensors; and
analyzing, using a machine learning, ML, analytics server implemented in the edge cloud network, the data collected using the plurality of sensors.
